(19) United States
(12) Patent Application Publication
(10) Pub. No.: US 2021/0113440 A1
(43) Pub. Date: Apr. 22, 2021

(54) USE OF TALC FOR PEARLESCENCE IN LIQUID COMPOSITIONS

(71) Applicant: IMERTECH SAS, Paris (FR)

(72) Inventor: Laure PAGIS, Toulouse (FR)

(21) Appl. No.: 16/493,937

(22) PCT Filed: Mar. 14, 2018
(86) PCT No.: PCT/EP2018/056398

§ 371 Date: Sep. 13, 2019

(30) Foreign Application Priority Data
Mar. 14, 2017 (EP) 17305271.3

(51) Int. Cl. A61K 8/25; A61Q 1/00; A61Q 19/10; C09C 1/00; C09C 1/28; C01B 33/22

(52) U.S. Cl. CPC A61K 8/25; A61Q 1/00; A61Q 19/10; C09C 1/0015; C09C 1/28; C01B 33/22; C01P 2006/64; C09C 2200/102; C01P 2004/51; C01P 2004/61; C01P 2006/12; C01P 2006/62; A61K 2800/436

(57) ABSTRACT

Use of a particulate talc material as a pearlescent agent in a liquid composition and method of increasing the pearlescence of a liquid composition by adding said particulate talc material, pearlescent liquid compositions comprising said particulate talc material and methods of making said pearlescent liquid compositions.

### TECHNICAL FIELD

The present invention relates generally to the use of a particulate talc mineral as a pearlescent agent in a liquid composition and methods of increasing the pearlescence of a liquid composition by adding a particulate talc material to said liquid composition. The present invention further relates to pearlescent liquid compositions comprising a particulate talc material and to methods of making said pearlescent liquid compositions.

### BACKGROUND

Pearlescence agents are widely used in liquid compositions, particularly liquid cosmetic compositions to provide the composition with an iridescent appearance and/or a nacreous or pearly lustre or sheen. This may, for example, give the compositions an appearance of richness and luxuriousness and make them more attractive to customers. Currently, the most commonly used pearlescence agents are synthetic substances such as fatty esters (e.g. glycol stearate) and fatty amides. However, synthetic pearlescence agents are not generally eco-friendly, may have an unpleasant odour, may cause an allergic reaction and/or may be toxic. Some natural substances such as mica have been used as pearlescence agents. However, these can be expensive and difficult to source and may also be difficult to maintain in suspension. It is therefore desirable to provide alternative and/or improved pearlescence agents for use in liquid compositions, particularly liquid cosmetic compositions.

### SUMMARY

In accordance with a first aspect of the present invention there is provided a use of a particulate talc material as a pearlescence agent in a liquid composition, wherein the particulate talc material has a lamellarity index equal to or greater than about 2.8.

In accordance with a second aspect of the present invention there is provided a method of increasing the pearlescence of a liquid composition, wherein the method comprises adding a particulate talc material having a lamellarity index equal to or greater than about 2.8 to the liquid composition.

In accordance with a third aspect of the present invention there is provided a pearlescent liquid composition comprising a particulate talc material having a lamellarity index equal to or greater than about 2.8.

In accordance with a fourth aspect of the present invention there is provided a method of making a pearlescent liquid composition, wherein the method comprises mixing a particulate talc material having a lamellarity index equal to or greater than about 2.8 with one or more components of the pearlescent liquid composition. The method may, for example, make a pearlescent liquid composition in accordance with any aspect or embodiment of the present invention.

In accordance with a sixth aspect of the present invention there is provided a pearlescent liquid composition obtained by and/or obtainable by any method or use of any aspect or embodiment of the present invention.

Certain embodiments of any aspect of the present invention may provide one or more of the following advantages:
improved pearlescent appearance (e.g. compared to the liquid composition without a pearlescence agent and/or compared to the liquid composition with a different pearlescence agent);
natural product;
does not cause allergic reaction;
non-toxic;
reduced amount required to obtain a desired pearlescence effect;
no malodour;
eco-friendly.

The details, examples and preferences provided in relation to any particulate one or more of the stated aspects of the present invention will be further described herein and apply equally to all aspects of the present invention. Any combination of the embodiments, examples and preferences described herein in all possible variations thereof is encompassed by the present invention unless otherwise indicated herein, or otherwise clearly contradicted by context.

### BRIEF DESCRIPTION OF THE FIGURES

FIG. 1 shows the pearlescence ranking of the compositions described in Example 1.

FIG. 2 shows the pearlescence ranking of the compositions described in Example 2.

FIG. 3 shows photographs of the compositions described in Example 2.

### DETAILED DESCRIPTION

There is provided herein a use of a particulate talc material as a pearlescence agent in a liquid composition.

There is also provided herein a method for increasing the pearlescence of a liquid composition, wherein the method comprises adding a particulate talc material to the liquid composition.

There is further provided herein a pearlescent liquid composition comprising a particulate talc material and a method of making such pearlescent liquid compositions.

The term “pearlescence agent” refers to any compound or material that can be used to give a composition to which it is added (e.g. a liquid composition) a pearlescent appearance. A “pearlescent appearance” refers to a pearl-like lustre or sheen that may also be referred to as nacreous and/or iridescence.

Pearlescence may, for example, be measured using a probe such as the Aello 1200™ probe and the dynamic extinction measurement technique wherein the signal fluctuation is evaluated and allowed to assess the pearlescent aspect. This gives a measurement of reflected light (%). This is described, for example, in Bolzinger et al., “Effects of surfactants on crystallization of ethylene glycol distearate in oil-in-water emulsion”, Colloids and Surfaces A: Physiochem. Eng. Aspects, 299, 2007, 93-100, the contents of which are incorporated herein by reference. A dispersion of mica (e.g. Timiron Starluster MP-115™ from Merck) may be used as a standard sample and assigned a pearlescence of 5. A suspension of EGDS crystallized in the absence of surfactant may be used as a standard sample and assigned a pearlescence of 0.

In certain embodiments, the % reflected light of the pearlescent liquid composition is at least about 40%. For example, the % reflected light of the pearlescent liquid composition is at least about 45% or at least about 50% or at least about 55% or at least about 60% or at least about 65% or at least about 70% or at least about 75%. In certain embodiments, the % reflected light of the pearlescent liquid composition is equal to or less than about 95%, for example equal to or less than about 90% or equal to or less than about 85% or equal to or less than about 80%.

In certain embodiments, pearlescence is determined by comparing the composition of and/or made by the invention with an identical composition that comprises a certain amount of a pearlescence agent used in industry instead of the particulate talc material described herein. For example, pearlescence may be determined by comparing the composition of and/or made by the invention with an identical composition that comprises 2 wt % glycol stearate in place of the particulate talc material having a lamellarity index equal to or greater than about 2.8.

“Particulate talc material” refers to particulate material made of hydrated magnesium silicate having the chemical formula H₂Mg₃(SiO₃)₄ or Mg₃Si₄O₁₀(OH)₂, or the mineral chlorite (hydrated magnesium aluminium silicate), a combination thereof, or a mineral substance derived therefrom and having similar properties.

When the particulate talc material is obtained from naturally occurring sources, it may be that some mineral impurities will inevitably contaminate the ground material. For example, naturally occurring talc may occur in association with other minerals such as dolomite. Also, in some circumstances, minor additions of other minerals may be included, for example, one or more of dolomite, kaolin, calcined kaolin, wollastonite, bauxite, or mica, could also be present. In general, however, the particulate mineral used in the invention will contain less than 5% by weight, for example less than 2 wt %, for example less than 1% by weight of other minerals.

In one embodiment, the particulate talc material undergoes minimal processing following mining or extraction. In a further embodiment, the particulate talc material is subjected to at least one physical modification process. The skilled artisan will readily know physical modification processes appropriate for use, which may be now known or hereafter discovered; appropriate physical modification processes include, but are not limited to, milling, drying, and air classifying. In yet another embodiment, the particulate talc material is subjected to at least one chemical modification process. The skilled artisan will readily know chemical modification processes appropriate for use in the present inventions, which may be now known or hereafter discovered; appropriate chemical modification processes include but are not limited to, silanization and calcination. The particulate talc material may, for example, be surface treated or surface untreated.

The surface treatment may, for example, serve to modify a property of the talc particulate and/or the liquid composition into which it is incorporated. In certain embodiments, the surface treatment enhances the spreadability of the composition and/or enhances the adhesion, water resistance, sebum absorption and/or surface smoothness of the composition, for example when applied to skin (e.g. human skin). The surface treatment may, for example, increase the hydrophobicity or lipophilicity of the talc particulate.

In certain embodiments, the surface treatment is an organosilane, an organophosphour, an organosulfur, or a mixture thereof. In certain embodiments, the surface treatment agent is a hydrocarbyl phosphoric acid, for example an alkyl phosphoric acid such as, for example a C₆-C₂₄ phosphonic acid, for example, n-octadecylphophonic acid. In certain embodiments, the surface treatment agent is a halo-alkyl phosphonic acid such as, for example, a fluoro-alkyl phosphonic acid such as, for example, nonafluoropentadecyl phosphonic acid.

In certain embodiments, the surface treatment is selected from one or more methicone, diemethicone, triethoxysilane, lauroyl lysine, C₉₋₁₅ flouroalcohol phosphates, magnesium myristate, triethoxy caprylsilane, polyhdyroxystearic acid and perflourooctyl triethoxysilane,

In certain embodiments, the surface treatment is or comprises microcrystalline cellulose.

In certain embodiments, the surface treatment is or comprises polyacrylate.

In certain embodiments, the surface treatment is or comprises a polyoxyalkylene (POA), for example, polyalkylene glycol (PAG) or polyalkylene oxide (PAO). As used herein, the term ‘polyalkylene glycol’ means a POA having a number average molecular mass below 20,000 g/mol, and the term ‘polyalkylene oxide’ means a POA having a number average molecular mass above 20,000 g/mol. In certain embodiments, the surface treatment comprises or is a polyalkylene glycol having a number average molecular mass of from about 100 to about 15,000 g/mol, for example, from about 200 to about 10,000 g/mol, or from about 500 to about 9000 g/mol, or from about 1000 to about 9000 g/mol, or from about 2000 to about 900 g/mol, or from about 4000 to about 9000 g/mol, or from about 6000 to about 9000 g/mol, or from about 6000 to about 8500 g/mol.

In certain embodiments, the surface treatment is or comprises a polyalkylene oxide selected from one or more of paraformaldehyde (polymethylene oxide), polytetramethylene glycol, polytetramehtylene ether glycol, polyethylene oxide, polypropylene oxide, polybutylene oxide, and combinations thereof.

In certain embodiments, the surface treatment is or comprises a polyethylene glycol. In certain embodiments, the surface treatment comprises or is a mixture of polyethylene glycol and polypropylene glycol (PPG).

In certain embodiments, the surface treatment is or comprises a polyether modified polysiloxane. The polyether modified polysiloxane may be derived from a linear polysiloxane. In certain embodiments, the polyether modified polysiloxane is derived from poly(dimethylsiloxane), poly(hexamethyldisiloxane), poly(octamethyltrisiloxane), poly(decamethyltetrasilozne), or combinations thereof.

In certain embodiments, the surface treatment comprises at least one siloxane. In general, siloxanes are any of a class of organic or inorganic chemical compounds comprising silicon, oxygen, and often carbon and hydrogen, based on the general empirical formula of R₂SiO, where R may be an alkyl group. Exemplary siloxanes include, but are not limited to, dimethylsiloxane, methylphenylsiloxane, methylhydrogen siloxane, methylhydrogen polysiloxane, methyltrimethoxysilane, octamethylcyclotetrasiloxane, hexamethyldisiloxane, diphenylsiloxane, and copolymers or blends of copolymers of any combination of monophenylsiloxaneunits, diphenylsiloxane units, phenylmethylsiloxane units, dimethylsiloxane units, monomethylsiloxane units, vinylsiloxane units, phenylvinylsiloxane units, methylvinylsiloxane units, ethylsiloxane units, phenylethylsiloxane units, ethylmethylsiloxane units, ethylvinylsiloxane units, or diethylsiloxane units.

In certain embodiments, the surface treatment comprises an amine, or an amine derivative. In certain embodiments, the surface treatment comprises an alkylated amine, for example, an alkylated alkyl amine such, as for example, an ethylated alkyl amine. In certain embodiments, the surface treatment comprises an alkoxylated amine, for example, an ethoxylated amine, or an alkoxylated alkyl amine, such as, for example, an ethoxylated alkyl amine.

In certain embodiments, the surface treatment comprises a polyalkylene glycol (PAG) and amine, for example, a PAG, an alkoxylated amine and a siloxane. In certain embodiments, the surface treatment comprises PAG (e.g., PEG), ethoxylated alkyl amine a siloxane. In certain embodiments, the surface treatment consists essentially or consists of the aforementioned surface treatment agents.

In certain embodiments, the surface treatment is present in an amount up to about 5 wt. %, based on the total weight of talc particulate, for example, from about 0.001 wt. % to about 5 wt. %, or from about 0.01 wt. % to about 2 wt. %, or from about 0.1 wt. % to about 2 wt. %, or from about 0.5 wt. % to about 1.5 wt. %, based on the total weight of talc particulate.

In certain embodiments, the particulate talc material is not surface treated.

The talc particulate material may, for example, be obtained from a natural source by grinding. For example, the talc particulate material may be obtained by or obtainable by delamination of talc suspended in a liquid.

Natural talc particulate is typically obtained by crushing and then grinding a mineral source of talc, which may be followed by a particle size classification step, in order to obtain a product having a desired particle size distribution. The particulate solid material may be ground autogenously, i.e. by attrition between the particles of the solid material themselves, or, alternatively, in the presence of a particulate grinding medium comprising particles of a different material from the talc particulate to be ground. These processes may be carried out with or without the presence of a dispersant and biocides, which may be added at any stage of the process.

The talc particulate may be prepared using techniques well known to a person of skill in the art, for example, techniques selected from comminution (e.g., crushing, grinding, milling), classification (e.g., hydrodynamic selection, screening and/or sieving) and drying.

In certain embodiments, the talc particulate is obtained and/or obtainable by a process according to that described in U.S. Pat. No. 6,348,536, the entire contents of which are hereby incorporated by reference.

More particularly, the talc particulate may be prepared by a process comprising:
(a) talc with a predetermined initial particle size is suspended in a liquid,
(b) the suspension is subjected to a delamination operation adapted so as to produce a separation of the leaves of the particles and so as to obtain a particle size less than the initial particle size,
(c) optionally subjecting the suspension to a selection as to eliminate particles with a size greater than a predetermined size,
(d) drying the suspension, and
(e) optionally treating the particles so as to limit the creation of strong bonds between them.

The starting talc is typically chosen having an initial particle size which is greater than the desired particle size. In certain embodiments, the starting talc is suspended in water in the presence of a dispersing agent such that the weight of dry matter based on the total weight of the suspension is from about 10% to about 60%. The suspension is typically homogenous. The grinding operation during delamination is, in certain embodiments, carried out as to obtain a d₅₀ₗₐₛₑᵣ of from about 10 μm to about 50 μm. The selection step may comprise hydrodynamic selection, which may be carried out in a turbine selector or in a hydrocyclone or in a centrifuge with an endless extraction screw. The suspension is advantageously dried in such a way as to reach a residual liquid level below 1%.

In certain embodiments, the talc particulate is prepared by a process comprising:
(a) delaminating a liquid suspension of a relatively coarse talc particulate having an initial particle size with a d₅₀ₗₐₛₑᵣ which is greater than a desired d₅₀ₗₐₛₑᵣ (e.g., greater than a desired d₅₀ₗₐₛₑᵣ of from about 10 μm to about 50 μm, or from about 10 μm to about 35 μm), to obtain a talc particulate having a particle size less than the initial particle size;
(b) at least partially drying the suspension thereby obtaining a talc particulate having the desired d₅₀ₗₐₛₑᵣ and optionally a desired lamellarity index.

In certain embodiments, the inorganic particulate, for example, talc particulate is not chemically treated during processing to obtain the desired particle size and lamellarity.

The particulate talc material may, for example, have an aluminium content equal to or less than about 20 wt % (based on the total weight of the particulate talc material). For example, the particulate talc material may have an aluminium content equal to or less than about 15 wt % or equal to or less than about 10 wt % or equal to or less than about 8 wt % or equal to or less than about 6 wt % or equal to or less than about 5 wt % or equal to or less than about 4 wt % or equal to or less than about 3 wt % or equal to or less than about 2 wt % or equal to or less than about 1.5 wt % or equal to or less than about 1 wt % or equal to or less than about 0.75 wt %. For example, the particulate talc material may have an aluminium content of at least about 0.1 wt % or at least about 0.2 wt % or at least about 0.4 wt %. For example, the particulate talc material may have an aluminium content ranging from about 0.1 wt % to about 5 wt % or from about 0.2 wt % to about 3 wt % or from about 0.4 wt % to about 2.5 wt %. The aluminium content is calculated as Al₂O₃ content, as may be determined by X-ray Fluorescence Spectroscopy (XFS).

The particulate talc material used in the present invention is a particulate talc material having a lamellarity index equal to or greater than about 2.8.

Lamellarity index characterizes the shape and flatness of particles (large dimension/thickness). The term “lamellarity index” is defined by the following ratio:

$$\frac{{d}_{50 laser} - {d}_{50 sedi}}{{d}_{50 sedi}}$$

in which “d₅₀ₗₐₛₑᵣ” is the value of the mean particle size (d₅₀) obtained using a laser particle size analyser as described above and “d_{50sedi}” is the value of the median diameter obtained by sedimentation using a sedigraph (standard ISO 13317-3), as described below. Reference may be made to the article by G. Baudet and J. P. Rona, Ind. Min. Mines et Carr. Les techn. June, July 1990, pp 55-61, which shows that this index is correlated to the mean ratio of the largest dimension of the particle to its smallest dimension.

In the sedimentation technique referred to above, particle size properties referred to herein for the talc particulate materials are as measured in a well-known manner by sedimentation of the particulate material in a fully dispersed condition in an aqueous medium using a Sedigraph 5100 machine as supplied by Micromeritics Instruments Corporation, Norcross, Ga., USA (www.micromeritics.com), referred to herein as a “Micromeritics Sedigraph 5100 unit”, and based on application of Stokes' Law. Such a machine provides measurements and a plot of the cumulative percentage by weight of particles having a size, referred to in the art as the ‘equivalent spherical diameter’ (e.s.d), less than given e.s.d values. The mean particle size d_{50sedi} is the value determined in this way of the particle e.s.d at which there are 50% by weight of the particles which have an equivalent spherical diameter less than that d₅₀ value. The d_{95sedi} value is the value at which 95% by weight of the particles have an esd less than that d_{95sedi} value. Particle size properties may be determined in accordance with ISO 13317-3, or any method equivalent thereto.

In the laser technique referred to above, particle size properties referred to herein for the particulate talc materials are measured by wet Malvern laser scattering (standard ISO 13320-1). In this technique, the size of particles in powders, suspensions and emulsions may be measured using the diffraction of a laser beam, based on the application of Mie theory. Such a machine, for example a Malvern Mastersizer S (as supplied by Malvern instruments) provides measurements and a plot of the cumulative percentage by volume of particles having a size, referred to in the art as the “equivalent spherical diameter” (e.s.d), less than given e.s.d values. The mean particle size d₅₀ is the value determined in this way of the particle e.s.d. at which there are 50% by weight of the particles which have an equivalent spherical diameter less than that d₅₀ value. For the avoidance of doubt, the measurement of particle size using laser light scattering is not an equivalent method to the sedimentation method referred to above.

In certain embodiments, the particulate talc material has a lamellarity index equal to or greater than about 2.9. For example, the particulate talc material may have a lamellarity index equal to or greater than about 3 or equal to or greater than about 3.1 or equal to or greater than about 3.2 or equal to or greater than about 3.3 or equal to or greater than about 3.4 or equal to or greater than about 3.5 or equal to or greater than about 3.6 or equal to or greater than about 3.7 or equal to or greater than about 3.8 or equal to or greater than about 3.9 or equal to or greater than about 4 or equal to or greater than about 4.1 or equal to or greater than about 4.2 or equal to or greater than about 4.3 or equal to or greater than about 4.4 or equal to or greater than about 4.5 or equal to or greater than about 4.6 or equal to or greater than about 4.7 or equal to or greater than about 4.8 or equal to or greater than about 4.9 or equal to or greater than about 5 or equal to or greater than about 5.1 or equal to or greater than about 5.2 or equal to or greater than about 5.3 or equal to or greater than about 5.4 or equal to or greater than about 5.5 or equal to or greater than about 5.6 or equal to or greater than about 5.7 or equal to or greater than about 5.8 or equal to or greater than about 5.9 or equal to or greater than about 6 or equal to or greater than about 6.1 or equal to or greater than about 6.2 or equal to or greater than about 6.3 or equal to or greater than about 6.4 or equal to or greater than about 6.5 or equal to or greater than about 6.6 or equal to or greater than about 6.7 or equal to or greater than about 6.8 or equal to or greater than about 6.9 or equal to or greater than about 7.

In certain embodiments, the particulate talc material has a lamellarity index equal to or less than about 20. For example, the particulate talc material may have a lamellarity index equal to or less than about 15 or equal to or less than about 10 or equal to or less than about 9.5 or equal to or less than about 9 or equal to or less than about 8.5 or equal to or less than about 8 or equal to or less than about 7.5.

The particulate talc material may, for example, have a d₅₀ (sedigraph) equal to or greater than about 1 μm. For example, the particulate talc material may have a d₅₀ (sedigraph) equal to or greater than about 1.1 μm or equal to or greater than about 1.2 μm or equal to or greater than about 1.3 μm or equal to or greater than about 1.4 μm or equal to or greater than about 1.5 μm or equal to or greater than about 1.6 μm or equal to or greater than about 1.7 μm or equal to or greater than about 1.8 μm or equal to or greater than about 1.9 μm or equal to or greater than about 2 μm. For example, the particulate talc material may have a d₅₀ (sedigraph) equal to or greater than about 2.1 μm or equal to or greater than about 2.2 μm or equal to or greater than about 2.3 μm or equal to or greater than about 2.4 μm or equal to or greater than about 2.5 μm.

The particulate talc material may, for example, have a d₅₀ (sedigraph) equal to or less than about 20 μm. For example, the particulate talc material may have a d₅₀ (sedigraph) equal to or less than about 19 μm or equal to or less than about 18 μm or equal to or less than about 17 μm or equal to or less than about 16 μm or equal to or less than about 15 μm or equal to or less than about 14 μm or equal to or less than about 13 μm or equal to or less than about 12 μm or equal to or less than about 11 μm or equal to or less than about 10 μm. For example, the particulate talc material may have a d₅₀ (sedigraph) equal to or less than about 9.5 μm or equal to or less than about 9 μm or equal to or less than about 8.5 μm or equal to less than about 8 μm or equal to or less than about 7.5 μm or equal to or less than about 7 μm or equal to or less than about 6.5 μm or equal to or less than about 6 μm or equal to or less than about 5.5 μm or equal to or less than about 5 μm or equal to or less than about 4.5 μm or equal to or less than about 4 μm or equal to or less than about 3.5 μm or equal to or less than about 3 μm.

In certain embodiments, the particulate talc material has a d₅₀ (sedigraph) ranging from about 1 μm to about 10 μm, for example from about 2 μm to about 8 μm. For example, the particulate talc material has a d₅₀ (sedigraph) ranging from about 1 μm to about 4 μm or from about 1.5 μm to about 3.5 μm or from about 2 μm to about 3 μm.

The particulate talc material may, for example, have a d₅₀ (laser) equal to or greater than about 5 μm. For example, the particulate talc material may have a d₅₀ (laser) equal to or greater than about 6 μm or equal to or greater than about 7 μm or equal to or greater than about 8 μm or equal to or greater than about 9 μm or equal to or greater than about 10 μm or equal to or greater than about 11 μm or equal to or greater than about 12 μm or equal to or greater than about 13 μm or equal to or greater than about 14 μm or equal to or greater than about 15 μm or equal to or greater than about 16 μm or equal to or greater than about 17 μm or equal to or greater than about 18 μm or equal to or greater than about 19 μm or equal to or greater than about 20 μm or equal to or greater than about 21 μm or equal to or greater than about 22 μm.

The particulate talc material may, for example, have a d₅₀ (laser) equal to or less than about 40 μm. For example, the particulate talc material may have a d₅₀ (laser) equal to or less than about 35 μm or equal to or less than about 30 μm or equal to or less than about 25 μm or equal to or less than about 24 μm or equal to or less than about 23 μm or equal to or less than about 22 μm or equal to or less than about 21 μm or equal to or less than about 20 μm or equal to or less than about 19 μm or equal to or less than about 18 μm or equal to or less than about 17 μm or equal to or less than about 16 μm or equal to or less than about 15 μm or equal to or less than about 14 μm or equal to or less than about 13 μm or equal to or less than about 12 μm or equal to or less than about 11 μm.

In certain embodiments, the particulate talc material has a d₅₀ (laser) ranging from about 5 μm to about 40 μm. For example, the particulate talc material may have a d₅₀ (laser) ranging from about 8 μm to about 35 μm or from about 10 μm to about 30 μm or from about 10 μm to about 25 μm. For example, the particulate talc material may have a d₅₀ (laser) ranging from about 5 μm to about 15 μm or from about 7 μm to about 14 μm or from about 8 μm to about 12 μm or from about 9 μm to about 11 μm. For example, the particulate talc material may have a d₅₀ (laser) ranging from about 15 μm to about 30 μm or from about 15 μm to about 25 μm or from about 18 μm to about 24 μm.

In certain embodiments, the particulate talc material has a d₁₀ (sedigraph) equal to or greater than about 0.05 μm. For example, the particulate talc material may have a d₁₀ (sedigraph) equal to or greater than about 0.1 μm or equal to or greater than about 0.15 μm or equal to or greater than about 0.2 μm or equal to or greater than about 0.25 μm or equal to or greater than about 0.3 μm or equal to or greater than about 0.35 μm or equal to or greater than about 0.4 μm.

In certain embodiments, the particulate talc material has a d₁₀ (sedigraph) equal to or less than about 2 μm. For example, the particulate talc material may have a d₁₀ (sedigraph) equal to or less than about 1.9 μm or equal to or less than about 1.8 μm or equal to or less than about 1.7 μm or equal to or less than about 1.6 μm or equal to or less than about 1.5 μm or equal to or less than about 1.4 μm or equal to or less than about 1.3 μm or equal to or less than about 1.2 μm or equal to or less than about 1.1. μm or equal to or less than about 1 μm or equal to or less than about 0.9 μm or equal to or less than about 0.8 μm or equal to or less than about 0.7 μm or equal to or less than about 0.6 μm.

In certain embodiments, the particulate talc material has a d₁₀ (sedigraph) ranging from about 0.05 μm to about 2 μm, for example from about 0.1 μm to about 1.5 μm, for example from about 0.2 μm to about 1 μm, for example from about 0.3 μm to about 0.6 μm.

In certain embodiments, the particulate talc material has a d₁₀ (laser) equal to or greater than about 2 μm. For example, the particulate talc material may have a d₁₀ (laser) equal to or greater than about 2.2 μm or equal to or greater than about 2.4 μm or equal to or greater than about 2.5 μm or equal to or greater than about 2.6 μm or equal to or greater than about 2.8 μm or equal to or greater than about 3 μm or equal to or greater than about 3.2 μm or equal to or greater than about 3.4 μm or equal to or greater than about 3.5 μm or equal to or greater than about 3.6 μm or equal to or greater than about 3.8 μm or equal to or greater than about 4 μm.

In certain embodiments, the particulate talc material has a d₁₀ (laser) equal to or less than about 10 μm. For example, the particulate talc material may have a d₁₀ (laser) equal to or less than about 9.5 μm or equal to or less than about 9 μm or equal to or less than about 8.5 μm or equal to or less than about 8 μm or equal to or less than about 7.5 μm or equal to or less than about 7 μm or equal to or less than about 6.5 μm or equal to or less than about 6 μm or equal to or less than about 5.5 μm or equal to or less than about 5 μm.

In certain embodiments, the particulate talc material has a d₁₀ (laser) ranging from about 2 μm to about 10 μm, for example from about 2.5 μm to about 8 μm, for example from about 3 μm to about 7 μm, for example from about 3.5 μm to about 6.5 μm.

In certain embodiments, the particulate talc material has a d₉₅ (sedigraph) equal to or greater than about 5 μm. For example, the particulate talc material may have a d₉₅ (sedigraph) equal to or greater than about 6.5 μm or equal to or greater than about 7 μm or equal to or greater than about 7.5 μm or equal to or greater than about 8 μm or equal to or greater than about 8.5 μm or equal to or greater than about 9 μm or equal to or greater than about 9.5 μm or equal to or greater than about 10 μm or equal to or greater than about 10.5 μm or equal to or greater than about 11 μm or equal to or greater than about 11.5 μm or equal to or greater than about 12 μm or equal to or greater than about 12.5 μm or equal to or greater than about 13 μm or equal to or greater than about 13.5 μm or equal to or greater than about 14 μm or equal to or greater than about 14.5 μm or equal to or greater than about 15 μm or equal to or greater than about 15.5 μm or equal to or greater than about 16 μm or equal to or greater than about 16.5 μm or equal to or greater than about 17 μm or equal to or greater than about 17.5 μm or equal to or greater than about 18 μm or equal to or greater than about 18.5 μm or equal to or greater than about 19 μm or equal to or greater than about 19.5 μm or equal to or greater than about 20 μm or equal to or greater than about 20.5 μm.

In certain embodiments, the particulate talc material has a d₉₅ (sedigraph) equal to or less than about 40 μm. For example, the particulate talc material may have a d₉₅ (sedigraph) equal to or less than about 38 μm or equal to or less than about 36 μm or equal to or less than about 35 μm or equal to or less than about 34 μm or equal to or less than about 32 μm or equal to or less than about 30 μm or equal to or less than about 28 μm or equal to or less than about 26 μm or equal to or less than about 25 μm or equal to or less than about 24 μm or equal to or less than about 22 μm or equal to or less than about 21 μm.

In certain embodiments, the particulate talc material has a d₉₅ (sedigraph) ranging from about 5 μm to about 40 μm, for example from about 6 μm to about 35 μm, for example from about 7 μm to about 30 μm, for example from about 10 μm to about 25 μm, for example from about 15 μm to about 22 μm.

In certain embodiments, the particulate talc material has a d₉₅ (laser) equal to or greater than about 20 μm. For example, the particulate talc material may have a d₉₅ (laser) equal to or greater than about 22 μm or equal to or greater than about 24 μm or equal to or greater than about 25 μm or equal to or greater than about 26 μm or equal to or greater than about 28 μm or equal to or greater than about 30 μm or equal to or greater than about 32 μm or equal to or greater than about 34 μm or equal to or greater than about 36 μm or equal to or greater than about 38 μm or equal to or greater than about 40 μm or equal to or greater than about 42 μm or equal to or greater than about 44 μm or equal to or greater than about 46 μm or equal to or greater than about 48 μm or equal to or greater than about 50 μm or equal to or greater than about 52 μm or equal to or greater than about 54 μm or equal to or greater than about 55 μm or equal to or greater than about 56 μm or equal to or greater than about 58 μm or equal to or greater than about 60 μm.

In certain embodiments, the particulate talc material has a d₉₅ (laser) equal to or less than about 80 μm. For example, the particulate talc material may have a d₉₅ (laser) equal to or less than about 78 μm or equal to or less than about 76 μm or equal to or less than about 75 μm or equal to or less than about 74 μm or equal to or less than about 72 μm or equal to or less than about 70 μm or equal to or less than about 68 μm or equal to or less than about 66 μm or equal to or less than about 65 μm or equal to or less than about 64 μm or equal to or less than about 62 μm.

In certain embodiments, the particulate talc material has a d₉₅ (laser) ranging from about 20 μm to about 80 μm, for example from about 25 μm to about 75 μm, for example from about 30 μm to about 70 μm, for example from about 40 μm to about 70 μm, for example from about 50 μm to about 70 μm, for example from about 55 μm to about 65 μm.

In certain embodiments, the particulate talc material has a d₁₀ (sedigraph) ranging from about 0.2 μm to about 0.8 μm and a d₅₀ (sedigraph) ranging from about 2 μm to about 3.5 μm. In certain embodiments, the particulate talc material further has a d₉₅ (sedigraph) ranging from about 5 μm to about 25 μm. In certain embodiments, the particulate talc material further has a d₉₅ (sedigraph) ranging from about 5 μm to about 15 μm and a lamellarity index ranging from about 3 to about 4. In certain embodiments, the particulate talc material further has a d₉₅ (sedigraph) ranging from about 15 μm to about 25 μm and a lamellarity index ranging from about 5 to about 9.

In certain embodiments, the particulate talc material has a d₁₀ (laser) ranging from about 2 μm to about 8 μm and a d₅₀ (laser) ranging from about 8 μm to about 25 μm. In certain embodiments, the particulate talc material further has a d₉₅ (laser) ranging from about 25 μm to about 65 μm. In certain embodiments, the particulate talc material has a d₅₀ (laser) ranging from about 8 μm to about 15 μm and a d₉₅ (laser) ranging from about 25 μm to about 35 μm and/or a lamellarity index ranging from about 3 to about 4. In certain embodiments, the particulate talc material has a d₅₀ (laser) ranging from about 15 μm to about 30 μm (e.g. from about 15 μm to about 25 μm) and a d₉₅ (laser) ranging from about 50 μm to about 70 μm (e.g. from about 55 μm to about 65 μm) and/or a lamellarity index ranging from about 5 to about 9.

As used herein, d₅₀ (sedigraph) and d₅₀ (laser) refer to the d₅₀ values measured respectively, according to the sedigraph or laser techniques described above.

The particulate talc material may, for example, have a BET surface area equal to or greater than about 10 m²/g. For example, the particulate talc material may have a BET surface area equal to or greater than about 11 m²/g or equal to or greater than about 12 m²/g or equal to or greater than about 13 m²/g or equal to or greater than about 14 m²/g or equal to or greater than about 15 m²/g.

The particulate talc material may, for example, have a BET surface area equal to or less than about 30 m²/g. For example, the particulate talc material may have a BET surface area equal to or less than about 25 m²/g or equal to or less than about 24 m²/g or equal to or less than about 23 m²/g or equal to or less than about 22 m²/g or equal to or less than about 21 m²/g or equal to or less than about 20 m²/g.

The particulate talc material may, for example, have a BET surface area ranging from about 10 m²/g to about 25 m²/g or from about 10 m²/g to about 20 m²/g.

As used herein, “BET surface area” refers to the area of the surface of the particles of the particulate talc material with respect to unit mass, determined according to the BET method by the quantity of nitrogen adsorbed on the surface of said particles so as to form a monomolecular layer completely covering said surface (measurement according to the BET method, AFNOR standard X11-621 and 622 or ISO 9277). In certain embodiments, BET surface area is determined in accordance with ISO 9277 or any method equivalent thereto.

In certain embodiments, the particulate talc material has a d₅₀ (sedigraph) ranging from about 1 μm to about 8 μm (e.g. from about 2 μm to about 8 μm) and BET surface area of at least about 10 m²/g. In certain embodiments, the particulate talc material has a d₅₀ (sedigraph) ranging from about 8 μm to about 15 μm and a BET surface area of at least about 5 m²/g. In certain embodiments, the particulate talc material has a d₅₀ (sedigraph) ranging from about 1 μm to about 8 μm (e.g. from about 2 μm to about 8 μm) and a lamellarity index of at least about 3.

The particulate talc material may, for example, have a Y value equal to or greater than about 75. For example, the particulate talc material may have a Y value equal to or greater than about 77.5 or equal to or greater than about 80 or equal to or greater than about 82 or equal to or greater than about 82.5 or equal to or greater than about 85 or equal to or greater than about 85.5 or equal to or greater than about 86 or equal to or greater than about 86.5 or equal to or greater than about 87 or equal to or greater than about 87.5 or equal to or greater than about 90. For example, the particulate talc material may have a Y value equal to or less than about 99, for example equal to or less than about 98 or equal to or less than about 96 or equal to or less than about 95. In certain embodiments, the particulate talc material has a L* equal to or greater than about 80. For example, the particulate talc material may have a L* value equal to or greater than about 81 or equal to or greater than about 82 or equal to or greater than about 83 or equal to or greater than about 84 or equal to or greater than about 85 or equal to or greater than about 86 or equal to or greater than about 87 or equal to or greater than about 88 or equal to or greater than about 89 or equal to or greater than about 90 or equal to or greater than about 91 or equal to or greater than about 92 or equal to or greater than about 93 or equal to or greater than about 94 or equal to or greater than about 95.

In certain embodiments, the particulate talc material has a L* equal to or less than about 100. For example, the particulate talc material may have a L* value equal to or less than about 99 or equal to or less than about 98 or equal to or less than about 97.

In certain embodiments, the particulate talc material has a b* equal to or less than about 2. For example, the particulate talc material may have a b* equal to or less than about 1.9 or equal to or less than about 1.8 or equal to or less than about 1.7 or equal to or less than about 1.6 or equal to or less than about 1.5 or equal to or less than about 1.6 or equal to or less than about 1.5 or equal to or less than about 1.4 or equal to or less than about 1.3 or equal to or less than about 1.2 or equal to or less than about 1.1 or equal to or less than about 1 or equal to or less than about 0.9 or equal to or less than about 0.8 or equal to or less than about 0.7 or equal to or less than about 0.6 or equal to or less than about 0.5. For example, the particulate talc material may have a b* equal to or greater than about −1.0 or equal to or greater than about −0.75 or equal to or greater than about −0.5 or equal to or greater than about −0.25.

L* whiteness and b* yellowness are determined using the L*a*b* colour space. Y is also determined using the L*a*b* colour space and may be referred to as whiteness (Minolta CR300, illuminant D65/2°). Y, L* whiteness and b* yellowness of the sheet may be measured using the spectrophotometer MINOLTA CM-3700D (illuminant D65/10°) from KONICA/MINOLTA.

In certain embodiments, the liquid composition is an ink and/or a paint. In certain embodiments, the liquid composition is a liquid soap. For example, the liquid soap may be dishwashing liquid, laundry detergent, body soap (e.g. shower or bath gel or cream), hand soap, face cleanser and/or hair shampoo.

In certain embodiments, the liquid composition is a liquid cosmetic composition. The term “cosmetic composition” means a composition which is compatible with the skin and/or hair, particularly the keratin-containing material making up the outer (uppermost) layer of human skin. In certain embodiments, its pH is balanced for use on the body (e.g. pH from about 4 to about 7). The cosmetic composition may be intended to be applied to the human body for cleansing, beautifying, promoting attractiveness, or altering the appearance without affecting the body's structure or functions. In certain embodiments, the cosmetic composition is a decorative cosmetic.

For example, the liquid cosmetic composition may be a liquid soap, hair conditioner, hair styling product, sun lotion, moisturizer (e.g. hand, body and/or foot moisturizer), eye cream, hand sanitizer, deodorant, lip salve, primer or make-up composition. The make-up composition may, for example, be a foundation, concealer, BB cream, CC cream, highlighter, blusher, eyeshadow, mascara, lip colouring (e.g. lip gloss), eyeliner, eyebrow applicator).

The liquid composition may, for example, comprise any base suitable for the intended purpose. In certain embodiments, the base is an oil and/or wax containing material. The base and, thus, the liquid composition, may comprise one or more other components such as one or more humectants, preservatives, emollients, fragrances and/or antioxidants.

The liquid soap (e.g. shower or bath gel or cream) may comprise a gel of water and detergent bases. The liquid soap may have other functional ingredients such as, for example, moisturizer/conditioner, colorants and/or fragrance. The term “gel” used herein includes a phase having gel-like properties, such as low or negligible flow on standing, or a liquid phase or low viscosity. For example, a gel may be a colloidal suspension of solids dispersed in a liquid or a sol. In certain embodiments, the gel has a Brookfield viscosity of at least about 0.5 Pa·s (at 100 rpm RV spindle 6) and optionally no greater than about 100 Pa·s (at 100 rpm and RV spindle 6). In certain embodiments, the gel is an emulsion of water and detergent base. The detergent base may comprise a surfactant or mixture of surfactants. In certain embodiments, the gel may be thixotropic (i.e. gel-like at rest but fluid when agitated (e.g. shaken or squeezed).

In certain embodiments, the base of the liquid composition is in the form of a liquid, gel, emulsion, lotion or paste. In certain embodiments, the base comprises or constitutes the components of the composition other than the particulate talc material.

The liquid composition may, for example, comprise water. Water may, for example, be present in an amount from about 10 wt % to about 95 wt % based on the total weight of the liquid composition. For example, water may be present in an amount of from about 20 wt % to about 90 wt % or from about 30 wt % to about 90 wt % or from about 40 wt % to about 80 wt % or from about 50 wt % to about 75 wt % or from about 50 wt % to about 70 wt %. The skilled person will be able to select suitable amounts of water for incorporation in the base, based on the amount of water in the final composition.

The liquid composition may comprise one or more surfactants. The one or more surfactants may, for example, be selected from zwitterionic, anionic, non-ionic and amphoteric surfactants, and mixtures thereof. Surfactant(s) may be present in the liquid composition in a total amount from about 1 wt % to about 60 wt % based on the total weight of the liquid composition. For example, surfactant(s) may be present in an amount ranging from about 5 wt % to about 50 wt % or from about 5 wt % to about 30 wt %. The skilled person will be able to select suitable amounts of surfactant for incorporation in the base, based on the amount of surfactant in the final composition.

Suitable zwitterionic surfactants include, but are not limited to, derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one substituent contains an anionic group, e.g., carboxy, sulfonate, sulfate, phosphate, or phosphonate. Illustrative zwitterionics are coco dimethyl carboxymethyl betaine, cocoamidopropyl betaine, cocobetaine, oleyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, lauryl bis-(2-hydroxypropyl)alpha-carboxyethyl betaine, and mixtures thereof. The sulfobetaines may include stearyl dimethyl sulfopropyl betaine, lauryl dimethyl sulfoethyl betaine, lauryl bis-(2-hydroxyethyl) sulfopropyl betaine and mixtures thereof.

Suitable anionic surfactants include, but are not limited to, ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium laureth sulfate, sodium lauryl sarcosinate, sodium lauroyl sarcosinate, potassium lauryl sulfate, sodium trideceth sulfate, sodium methyl lauroyl taurate, sodium lauroyl isethionate, sodium laureth sulfosuccinate, sodium lauroyl sulfosuccinate, sodium tridecyl benzene sulfonate, sodium dodecyl benzene sulfonate, sodium lauryl amphoacetate, sodium lauryl sulfoacetate, sodium cocoyl isethionate, sodium methyl cocoyl taurate and mixtures thereof. The anionic surfactant may be, for example, an aliphatic sulfonate, such as a primary C₈-C₂₂ alkane sulfonate, primary C₈-C₂₂ alkane disulfonate, C₈-C₂₂ alkene sulfonate, C₈-C₂₂ hydroxyalkane sulfonate or alkyl glyceryl ether sulfonate.

Suitable non-ionic surfactants include the reaction products of compounds having a hydrophobic group and a reactive hydrogen atom. These include alcohols, acids, amides or alkyl phenols reacted with alkylene oxides, especially ethylene oxide either alone or with propylene oxide. Exemplary non-ionics are C₆-C₂₂ alkyl phenols-ethylene oxide condensates, the condensation products of C₈-C₁₈ aliphatic primary or secondary linear or branched alcohols with ethylene oxide, and products made by condensation of ethylene oxide with the reaction products of propylene oxide and ethylenediamine. Other nonionics include long chain tertiary amine oxides, long chain tertiary phosphine oxides and dialkyl sulphoxides. Other non-ionics are surfactants based on cocoamide and produced by reacting cocoamide with an alcohol amine, such as ethanolamine. Exemplary non-ionics include cocoamide MEA and cocoamide DEA. Other suitable non-ionics include alkyl polyglucosides such as decyl glucoside, lauryl glucoside and octyl glucoside. Also useful are the alkyl polysaccharides.

Suitable cationic surfactants include, but are not limited, to octenidine dihydrochloride, permanently charged quaternary ammonium surfactants such as alkyltrimethylammonium salts (e.g., cetyl trimethylammonium bromide, cetyl trimethylammonium chloride), cetylpyridinium chloride, benzalkonium chloride, benzethonium chloride, 5-Bromo-5-nitro-1,3-dioxane, dimethyldioctadecylammonium chloride, cetrimonium bromide and dioctadecyldimethylammonium bromide.

These surfactants serve primarily as a cleansing agent, i.e., constituting or forming part of the detergent component of the composition. These surfactants may comprise up to about 50 wt. % of the liquid composition, based on the total weight of the liquid composition, for example, from about 1 wt. % to about 45 wt. % of the liquid composition, or at least about 5 wt. %, or at least about 10 wt. %, or at least about 15 wt. %, or at least about 20 wt. %, or at least about 25 wt. % of the liquid composition.

In certain embodiments, the liquid composition comprises one or more thickening agents or suspending agents (e.g. rheology modifier). Such agents may enhance the stability of the inorganic particulate material dispersed throughout the gel. Suitable thickening agents include water soluble/dispersable polymers, which may be cationic, anionic, amphoteric or non-ionic with molecular weights typically greater than about 100,000 Daltons. Such agents may also serve to increase the viscosity of the liquid composition. Exemplary thickening or suspending agents include carbohydrate gums such as cellulose gum, microcrystalline cellulose, cellulose gel, hydroxyethyl cellulose, hydroxypropyl cellulose, sodium carboxymethylcellulose, methyl cellulose, ethyl cellulose, guar gum, gum karaya, gum tragacanth, gum arabic, gum acacia, gum agar, xanthan gum and mixtures thereof; modified and nonmodified starch granules and pregelatinized cold water soluble starch; emulsion polymers; cationic polymer such as modified polysaccharides; cationic modified cellulose; synthetic cationic polymer; cationic starches; cationic galactomannans; and high molecular weight polyethylene glycols, esters of ethylene glycol or esters of polyethylene glycol. Other suitable thickening/suspending agents include for example polyacrylic acid, copolymers and cross-linked polymers of acrylic acid, copolymers of acrylic acid with a hydrophobic monomer, copolymers of carboxylic acid-containing monomers and acrylic esters, cross-linked copolymers of acrylic acid and acrylate esters.

A thickening agent or suspending agent, such as a rheology modifier, when present, may be present in a total amount of from about 0.1 wt. % to about 50 wt. % by weight, based on the total weight of the liquid composition, for example, from about 0.1 wt. % to about 35 wt. %, or from about 0.1 wt. % to about 20 wt. %, or from about 0.1 wt. % to about 10 wt. %, or from about 0.1 wt. % to about 5 wt. %. The skilled person will be able to select suitable amounts of each component for incorporation in the base, based on the amount of the component in the final composition.

The liquid composition may contain other components conventionally found in cosmetic applications for skin and hair, including, without limitation, skin conditioning/moisturising agents, hair conditioning/moisturising agents perfumes, fragrances, opacifiers, pearlescing agents, colourings, preservatives, chelating agents, humectants, herb and/or plant extracts, essential oils, proteins, pH adjusting agents, and anti-microbials. The total amount of other components may be present in amount of from about 0.1 to about 30 wt. %, based on the total weight of the liquid composition, for example, from about 0.1 wt. % to about 20 wt. %, or from about 0.1 wt. % to about 15 wt. %, or from about 0.5 wt. % to about 10 wt. %, or from about 1 wt. % to about 10 wt. %, or from about 1 wt. % about 5 wt. %. The skilled person will be able to select suitable amounts of each component for incorporation in the base, based on the amount of the component in the final composition. A suitable amount of pH adjusting agent may be added, if necessary, to adjust the pH of the composition, particularly if the composition is a personal care composition. Suitable pH adjusting agents in sodium hydroxide, sodium chloride and citric acid.

In certain embodiments, the composition is a liquid preparation, for example in the form of an, elixir, syrup, suspension, spray, emulsion, lotion, gel, cream and solutions. For example, in certain embodiments the composition is in the form of a gel, cream, lotion or emulsion. Techniques and formulations generally may be found in Remington, The Science and Practice of Pharmacy, Mack Publishing Co., Easton, Pa., latest edition.

The composition may further contain ingredients selected from, for example, absorbents, excipients, diluents, carriers, adjuvants, excipients, vehicles, preserving agents, fillers, hydrating agents, binders, colouring agents, disintegrating agents, wetting agents, emollients, emulsifying agents, suspending agents, sweetening agents, flavouring agents, perfuming agents, antibacterial agents, antifungal agents, antioxidants, cleansing agents, exfoliating agents, lubricating agents, texture enhancing agents, coating agents, encapsulating agents, film-forming agents, thickening agents and dispersing agents, depending on the nature of the mode of administration and dosage forms. One or more (e.g. all) of the further ingredients may, for example, be skin-compatible in that they do not have any adverse effect when applied to the skin.

In certain embodiments, the liquid composition is substantially free of synthetic pearlescence agents. In certain embodiments, the liquid composition is substantially free of pearlescence agents other than the particulate talc material (having a lamellarity index equal to or greater than about 2.8).

The term “synthetic pearlescence agents” refers to pearlescence agents such as fatty esters (e.g. glycol stearates) and fatty amides that are not naturally occurring. The term “substantially” in this context means that the synthetic pearlescence agents are not present in the liquid composition in an amount that provides a pearlescent appearance. For example, the term “substantially free” may mean that the liquid composition comprises equal to or less than about 3 wt % or equal to or less than about 2 wt % or equal to or less than about 1.5 wt % or equal to or less than about 1 wt % or equal to or less than about 0.5 wt % or equal to or less than about 0.2 wt % of synthetic pearlescence agents/other pearlescence agents.

The particulate talc material may be used in any effective amount to provide the liquid composition with a pearlescence effect. The total amount of the particulate talc material in the liquid composition may be varied depending on the nature of the liquid composition and the desired pearlescence effect to be obtained. In certain embodiments, the total amount of the particulate talc material in the liquid composition is at least about 0.1 wt % (based on the total weight of the liquid composition). For example, the total amount of the particulate talc material in the liquid composition may be at least about 0.2 wt % or at least about 0.3 wt % or at least about 0.4 wt % or at least about 0.5 wt %. In certain embodiments, the total amount of the particulate talc material in the liquid composition is equal to or less than about 10 wt %. For example, the total amount of the particulate talc material in the liquid composition may be equal to or less than about 9 wt % or equal to or less than about 8 wt % or equal to or less than about 7 wt % or equal to or less than about 6 wt % or equal to or less than about 5 wt % or equal to or less than about 4 wt % or equal to or less than about 3 wt % or equal to or less than about 2 wt %. In certain embodiments, the total amount of the particulate talc material in the liquid composition ranges from about 0.1 wt % to about 10 wt % or from about 0.1 wt % to about 5 wt % or from about 0.2 wt % to about 4 wt % or from about 0.2 wt % to about 2 wt % or from about 0.5 wt % to about 1.5 wt %.

The liquid composition may, for example, have a L* equal to or greater than about 60. For example, the liquid composition may have a L* equal to or greater than about 65 or equal to or greater than about 70 or equal to or greater than about 75 or equal to or greater than about 80 or equal to or greater than about 85 or equal to or greater than about 90 or equal to or greater than about 95. For example, the liquid composition may have a L* equal to or less than about 100 or equal to or less than about 99 or equal to or less than about 98 or equal to or less than about 97.

The liquid composition may, for example, have a b* equal to or less than about 2. For example, the liquid composition may have a b* equal to or less than about 1.5 or equal to or less than about 1 or equal to or less than about 0.5 or equal to or less than about 0.2 or equal to or less than about 0. The liquid composition may, for example, have a b* equal to or greater than about −1 or equal to or greater than about 0.5 or equal to or greater than about 0.25.

The liquid composition may, for example, have a pH ranging from about 4 to about 8. For example, the liquid composition may have a pH ranging from about 4.5 to about 7.5 or from about 4.5 to about 6.5 or from about 5 to about 8 or from about 5.5 to about 7.5 or from about 6 to about 8 or from about 6.5 to about 7.5.

The liquid composition may, for example, be stable for at least 3 months at 45° C. For example, the liquid composition may be stable for at least 4 months at 45° C. or at least about 5 months at 45° C. or at least about 6 months at 45° C. Stability may, for example, be observed as no sedimentation when centrifuged at 1600 rpm for 15 minutes.

The liquid composition may be prepared by any suitable or conventional method well known to those skilled in the art. Such methods may, for example, generally comprise combining the components of the liquid composition, for example in a liquid, slurry or slurry form. The components of the liquid composition may be combined in any suitable order to make the liquid composition. The particulate talc material may be mixed with any one or more of the components of the liquid composition during the method of making the liquid composition. The method may, for example, comprise mixing and optionally milling the mixture of components and then forming the cosmetic composition therefrom. The components may, for example, be brought together in a blender or other mixing apparatus under conditions of suitably low shear so as to preserve the inherent properties of the particulate material.

### EXAMPLES

Experiments were performed to measure the capacity of particulate talc materials to act as a pearlescence agent in a liquid composition.

### Example 1

Compositions comprising a transparent commercial shower gel base (Sanex® Zero) with 0.75 wt % of one of the particulate talc materials specified in Table 1 below were ranked by 10 different people in order of pearlescence. The compositions were also compared to one of the commercial cosmetic compositions Gliss™ (Ultimate Color Shampoo) and Ushuaia™ (Rltuels d'Asie Shampooing Douche Homme Effet Glacant Roche Volcanique).

TABLE 1

| | d₅₀ (sedi) | d₅₀ (laser) | Lamellarity | | | |
|---|---|---|---|---|---|---|
| Talc | (μm) | (μm) | index | Y | L* | b* |
| 1 | 1.3 | 4.2 | 2.2 | 94.3 | 97.8 | 0.25 |
| 2 | 0.7 | 3 | 3.3 | 83.5 | 93.2 | 0.30 |
| 3 | 1.6 | 4.7 | 1.9 | 82.0 | 92.6 | 0.62 |
| 4 | 2.8 | 19 | 5.8 | 89.9 | 95.5 | 0.97 |
| 5 | 2.9 | 23.7 | 7.2 | 87.5 | 95.0 | 0.67 |
| 6 | 2.3 | 12.7 | 4.5 | 92.3 | 96.9 | 1.21 |
| 7 | 1.3 | 4.2 | 2.2 | 94.4 | 97.8 | 0.80 |
| 8 | 2.1 | 6.3 | 2.0 | 94.6 | 97.9 | 0.33 |
| 9 | 11.3 | 17.3 | 0.5 | 89.1 | 95.6 | 0.34 |
| 10 | 1.3 | 4.2 | 2.2 | 88.1 | 95.2 | −0.02 |
| 11 | 2.3 | 10.3 | 3.5 | 82.7 | 91.9 | 0.02 |
| 12 | 0.5 | 2.4 | 3.8 | 92.2 | 96.9 | 0.81 |
| 13 | 10.1 | 14.9 | 0.5 | 87.3 | 94.9 | 0.87 |

The results are shown in FIG. 1. It was surprisingly found that particulate talc materials with a high lamellarity index provide a better pearlescence effect.

### Example 2

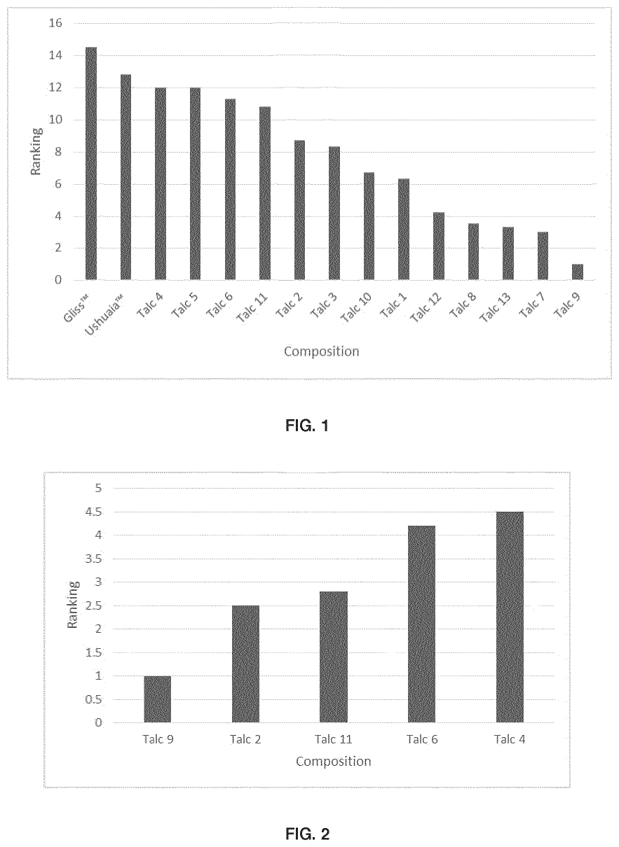

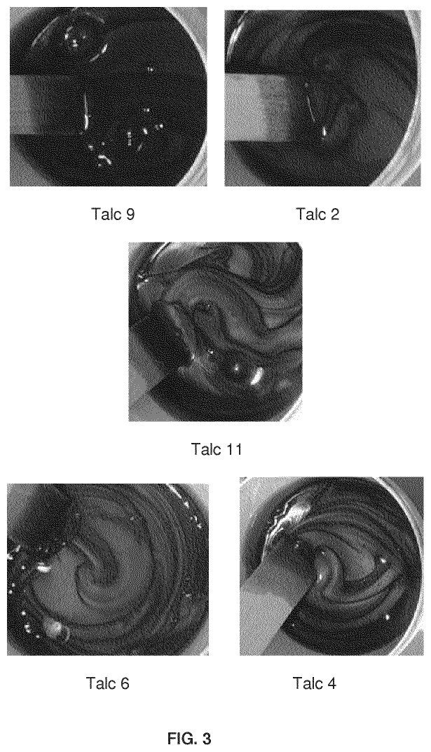

Compositions comprising a transparent commercial shower gel base (Sanex® Zero) with 0.75 wt % of one of particulate talc materials 2, 4, 6, 9 and 11 (as specified in Table 1 above) and 0.3 wt % of a black pigment (GLW60GBSP, which contains 58 wt %-62 wt % iron oxides, available from Kobo Products, Inc) were prepared. The compositions were ranked in order of pearlescence by 10 people. The results are shown in FIGS. 2 and 3. Again, it was surprisingly found that particulate talc materials with a high lamellarity index provide a better pearlescence effect.

### Example 3

Compositions comprising a transparent commercial shower gel base (Sanex® Zero or Timotei® Pure) and different amounts of the particulate Talc 6 specified in Table 1 above and optionally 0.3 wt % of the black pigment described in Example 2 were prepared. Comparative compositions comprising a transparent commercial shower gel base Sanex® Zero or Timotei® pure) and different amounts of benchmark pearlescence agents and optionally 40 wt % of the black pigment described in Example 2 were also prepared. The benchmark pearlescence agents were benchmark 1 (glycol distearate and coco-glucoside and glycerine), benchmark 2 (glycol distearate and sodium laureth sulfate and cocamide MEA), benchmark 3 (lauryl glucoside and stearyl citrate) (and benchmark 4 (sodium laureth sulfate/glycereth-2 cocoate/glycol distearate) (obtained from Kao). These compositions were ranked in order of pearlescence by 6 people.

The compositions using the Timotei® Pure base were ranked as follows (less pearlescent to more pearlescent):
4 wt % and 10 wt % Talc 6<0.7 wt % Talc 6<0.5 wt % Talc 6<0.75 wt % and 1 wt % Talc 6<2 wt % Talc 6<0.6 wt % Talc 6<4 wt % benchmark 3<3 wt % benchmark 1<1.5 wt % Talc 6<4 wt % benchmark 4<3 wt % benchmark 2.

There was only a small difference between 4 wt % and 10 wt % Talc 6 and 2 wt % Talc 6.

The compositions using the Sanex® Zero base were ranked as follows (less pearlescent to more pearlescent):
4 wt % benchmark 3<3 wt % benchmark 1<0.6 wt % Talc 6<1.5 wt % Talc 6.

The compositions using the Sanex® Zero base and 40 wt % black pigment were ranked as follows (less pearlescent to more pearlescent):
0.5 wt % Talc 6<4 wt % benchmark 3<0.6 wt % Talc 6<3 wt % benchmark 1<0.7 wt % Talc 6<0.75 wt % Talc 6<1 wt % Talc 6<3 wt % benchmark 2<4 wt % benchmark 4<1.5 wt % Talc 6 and 2 wt % Talc 6<10 wt % Talc 6.

### Example 4

Compositions were prepared in the following typical shower gel formulation (QSP=quantity sufficient for).

| Phase | INCI Name | % |
|---|---|---|
| A | Aqua | QSP 100 |
| | Acrylates copolymer | 5.0 |
| | Glycerin | 1.0 |
| B | Sodium laureth sulfate | 45.0 |
| | Sodium hydroxide | 3.0 |
| C | Aqua (and) methylisothiazolinone | 0.1 |
| | Sodium chloride | 2.0 |
| D | Aqua | 0.1 |
| | Glycerin | 2.0 |
| | Pearlising agent | x |
| E | Citric add (50% in a water solution) | QSP pH 5.5 |

The phase A ingredients were mixed until homogenous then the phase B ingredients were mixed successively into phase A. The composition was neutralized with sodium hydroxide (solution at 10%) and mixed until homogenous. Phase C ingredients were stirred in one after another. Phase D ingredients were pre-mixed and then added to the blend. The pH was adjusted using phase E if necessary.

To measure pearlescence intensity, a sensory analysis was conducted with 20 volunteers according to ISO 13299. Volunteers assessed the pearlescent intensity of the formulations on a scale of 0 to 10.

The pearlising agent was either 1% of a talc having a d₅₀(laser) of 11 and a d₅₀(sedi) of 2 and a lamellarity index of 4.5, or 3% of a synthetic pearlising agent (30% glycol distearate in a surfactant solution).

The panel test results showed that the formulation comprising the talc pearlising agent had a greater pearlescent effect than the formulation comprising the glycol distearate pearlising agent (result of 9.2 vs a result of 7.2). Therefore, the new talc pearlising agent can be used to replace conventional synthetic pearlising agents.

The foregoing broadly describes certain embodiments of the present invention without limitation. Variations and modifications as will be readily apparent to those skilled in the art are intended to be within the scope of the present invention as defined in and by the appended claims.

The following numbered paragraphs define particular embodiments of the present invention:
1. Use of a particulate talc material as a pearlescence agent in a liquid composition, wherein the particulate talc material has a lamellarity index equal to or greater than about 2.8.
2. A method of increasing the pearlescence of a liquid composition, the method comprising adding a particulate talc material having a lamellarity index equal to or greater than about 2.8 to the liquid composition.
3. The use of paragraph 1 or the method of paragraph 2, wherein the particulate talc material has a lamellarity index equal to or greater than about 3, for example equal to or greater than about 3.5 or equal to or greater than about 4.
4. The use or method of any preceding paragraph, wherein the particulate talc material has a d₅₀ (sedigraph) equal to or greater than about 1 μm, for example equal to or greater than about 1.5 μm or equal to or greater than about 2 μm.
5. The use or method of any preceding paragraph, wherein the particulate talc material has a d₅₀ (sedigraph) equal to or less than about 20 μm, for example equal to or less than about 10 μm or equal to or less than about 5 μm.
6. The use or method of any preceding paragraph, wherein the particulate talc material has a d₅₀ (laser) equal to or greater than about 5 μm, for example equal to or greater than about 10 μm or equal to or greater than about 15 μm.
7. The use or method of any preceding paragraph, wherein the particulate talc material has a d₅₀ (laser) equal to or less than about 40 μm, for example equal to or less than about 35 μm or equal to or less than about 30 μm.
8. The use or method of any preceding paragraph, wherein the particulate talc material has a BET surface area equal to or greater than about 10 m²/g, for example equal to or greater than about 15 m²/g.
9. The use or method of any preceding paragraph, wherein the particulate talc material has a Y value equal to or greater than about 75, for example equal to or greater than about 80 or equal to or greater than about 85.
10. The use or method of any preceding paragraph, wherein the particulate talc material has a L* equal to or greater than about 80, for example equal to or greater than about 85.
11. The use or method of any preceding paragraph, wherein the particulate talc material has a b* equal to or less than about 2, for example equal to or less than about 1.
12. The use or method of any preceding paragraph, wherein the particulate talc material is obtained or obtainable by delamination of a talc suspended in a liquid.
13. The use or method of any preceding paragraph, wherein the liquid composition is a liquid soap or a liquid make-up composition.
14. The use or method of any preceding paragraph, wherein the liquid composition is substantially free of synthetic pearlescent agents.
15. The use or method of any preceding paragraph, wherein the liquid composition is substantially free of pearlescent agents other than the particulate talc material having a lamellarity index equal to or greater than about 2.8.
16. The use or method of any preceding paragraph, wherein the liquid composition has a % reflected light of at least about 40%, for example at least about 50% or at least about 60%.
17. A pearlescent liquid composition comprising a particulate talc material having a lamellarity index equal to or greater than about 2.8
18. The pearlescent liquid composition of paragraph 17, wherein the composition is substantially free of synthetic pearlescent agents.
19. The pearlescent liquid composition of paragraph 17 or 18, wherein the composition is substantially free of pearlescent agents other than the particulate talc material having a lamellarity index equal to or greater than about 2.8.
20. The pearlescent liquid composition of any one of paragraphs 17 to 19, wherein the particulate talc material has a lamellarity index equal to or greater than about 3, for example equal to or greater than about 3.5 or equal to or greater than about 4.
21. The pearlescent liquid composition of any one of paragraphs 17 to 20, wherein the particulate talc material has a d₅₀ (sedigraph) equal to or greater than about 1 μm, for example equal to or greater than about 1.5 μm or equal to or greater than about 2 μm.
22. The pearlescent liquid composition of any one of paragraphs 17 to 21, wherein the particulate talc material has a d₅₀ (sedigraph) equal to or less than about 20 μm, for example equal to or less than about 10 μm or equal to or less than about 5 μm.
23. The pearlescent liquid composition of any one of paragraphs 17 to 22, wherein the particulate talc material has a d₅₀ (laser) equal to or greater than about 5 μm, for example equal to or greater than about 10 μm or equal to or greater than about 15 μm.
24. The pearlescent liquid composition of any one of paragraphs 17 to 23, wherein the particulate talc material has a d₅₀ (laser) equal to or less than about 40 μm, for example equal to or less than about 35 μm or equal to or less than about 30 μm.
25. The pearlescent liquid composition of any one of paragraphs 17 to 24, wherein the particulate talc material has a BET surface area equal to or greater than about 10 m²/g, for example equal to or greater than about 15 m2/g.
26. The pearlescent liquid composition of any one of paragraphs 17 to 25, wherein the particulate talc material has a Y value equal to or greater than about 75, for example equal to or greater than about 80 or equal to or greater than about 85.
27. The pearlescent liquid composition of any one of paragraphs 17 to 26, wherein the particulate talc material has a L* equal to or greater than about 80, for example equal to or greater than about 85.
28. The pearlescent liquid composition of any one of paragraphs 17 to 27, wherein the particulate talc material has a b* equal to or less than about 2, for example equal to or less than about 1.
29. The pearlescent liquid composition of any one of paragraphs 17 to 28, wherein the particulate talc material is obtained or obtainable by delamination of a talc suspended in a liquid.
30. The pearlescent liquid composition of any one of paragraphs 17 to 29, wherein the liquid composition is a liquid soap or a liquid make-up composition.
31. The pearlescent liquid composition of any one of paragraphs 17 to 30, wherein the pearlescent liquid composition has a % reflected light of at least about 40%, for example at least about 50% or at least about 60%.
32. The pearlescent liquid composition of any one of paragraphs 17 to 31, wherein the pearlescent liquid composition comprises from about 0.1 wt % to about 10 wt % of the particulate talc material, for example from about 0.2 wt % to about 5 wt % or from about 0.5 wt % to about 1.5 wt % of the particulate talc material.
33. A method of making a pearlescent liquid composition of any one of paragraphs 17 to 31, the method comprising mixing a particulate talc material having a lamellarity index equal to or greater than about 2.8 with one or more components of the pearlescent liquid composition.

1. (canceled)

2. A method of increasing the pearlescence of a liquid composition, the method comprising adding a particulate talc material having a lamellarity index equal to or greater than about 2.8 to the liquid composition.

3. The method of claim 2, wherein the particulate talc material has a lamellarity index equal to or greater than about 3.

4. The method claim 2, wherein the particulate talc material has a d₅₀ (sedigraph) (a) equal to or greater than about 1 μm.

5. The method of claim 2, wherein the particulate talc material has a d₅₀ (laser) (a) equal to or greater than about 5 μm, and (b) equal to or less than about 40 μm.

6. The method of claim 2, wherein the particulate talc material has a BET surface area equal to or greater than about 10 m²/g.

7. The method of claim 2, wherein the particulate talc material has:
a Y value equal to or greater than about 75,
a L* equal to or greater than about 80,
a b* equal to or less than about 2.

8. The method of claim 2, wherein the particulate talc material is obtained or obtainable by delamination of a talc suspended in a liquid.

9. The use or method of claim 2, wherein the liquid composition is a liquid soap or a liquid make-up composition.

10. The use or method of claim 2, wherein the liquid composition is substantially free of synthetic pearlescent agents.

11. The method of claim 2, wherein the liquid composition is substantially free of pearlescent agents other than the particulate talc material.

12. The method of claim 2, wherein the liquid composition has a % reflected light of at least about 40%.

13. A pearlescent liquid composition comprising a particulate talc material having a lamellarity index equal to or greater than about 2.8.

14. (canceled)

15. A method of making a pearlescent liquid composition of of claim 13, the method comprising mixing a particulate talc material having a lamellarity index equal to or greater than about 2.8 with one or more components of the pearlescent liquid composition.

16. The method of claim 2 wherein the particulate talc material has a L* equal to or greater than about 80.

17. The method of claim 2 wherein the particulate talc material has a b* equal to or less than about 2.

18. The method of claim 5 wherein the particulate talc material has a BET surface area equal to or greater than about 10 m2/g.

19. The method of claim 18, wherein the particulate talc material has:
a Y value equal to or greater than about 75;
a L* equal to or greater than about 80; and
a b* equal to or less than about 2

20. The method of claim 19, wherein the liquid composition is substantially free of pearlescent agents other than the particulate talc material.

21. The method of claim 20, wherein the liquid composition has a % reflected light of at least about 40%.

22. The method of claim 21, wherein the particulate talc material has a lamellarity index equal to or greater than about 3.5.
